SEQUENCE LISTING (29 sequences)

SEQ ID NO: 1 - DNA, 39 bp, Artificial Sequence
  Description of Artificial Sequence Synthetic primer
tttctcgagatgagacgctacaagctctttctcatgttc

SEQ ID NO: 2 - DNA, 97 bp, Artificial Sequence
  Description of Artificial Sequence Synthetic primer
atgagacgctacaagctctttctcatgttctgtatggccggcctgtgcctcatctccttcctgcacttcttcaagaccctgtcctatgtcaccttcc

SEQ ID NO: 3 - DNA, 100 bp, Artificial Sequence
  Description of Artificial Sequence Synthetic primer
cctgtcctatgtcaccttcccacgagaactggcctccctcagccctaacctggtgtccagctttttctggaacaatgccccggtcacgccccaggccagc

SEQ ID NO: 4 - DNA, 102 bp, Artificial Sequence
  Description of Artificial Sequence Synthetic primer
cggtcacgccccaggccagccctgagccaggaggccctgacctgctgcgtaccccactctactcccactcgcccctgctgcagccgctgccgcccagcaagg

SEQ ID NO: 5 - DNA, 93 bp, Artificial Sequence
  Description of Artificial Sequence Synthetic primer
agccgctgccgcccagcaaggcggccgaggagctccaccgggtggacttggtgctgcccgaggacaccaccgagtatttcgtgcgcaccaagg

SEQ ID NO: 6 - DNA, 98 bp, Artificial Sequence
  Description of Artificial Sequence Synthetic primer
gtatttcgtgcgcaccaaggctggaggcgtctgcttcaaacccggcaccaagatgctggagagaccgcctccgggacgaccggaggagaagcctgagg

SEQ ID NO: 7 - DNA, 83 bp, Artificial Sequence
  Description of Artificial Sequence Synthetic primer
accggaggagaagcctgagggggccaacggatcctcggcccggcgaccaccccggtacctcctgagcgcccgggagcgcacgg

SEQ ID NO: 8 - DNA, 104 bp, Artificial Sequence
  Description of Artificial Sequence Synthetic primer
gagcgcccgggagcgcacggggggccgaggtgcacgacgcaagtgggtggagtgcgtgtgtctgcccggatggcacggacccagctgcggcgtgcccactgtgg

SEQ ID NO: 9 - DNA, 84 bp, Artificial Sequence
  Description of Artificial Sequence Synthetic primer
agctgcggcgtgcccactgtggtgcagtattccaacctgcctaccaaggagcggctggtgcccagggaggtgccgcgccgcgtc

SEQ ID NO: 10 - DNA, 99 bp, Artificial Sequence
  Description of Artificial Sequence Synthetic primer
agggaggtgccgcgccgcgtcattaatgctatcaacgtcaaccacgagttcgacctgctggacgtgcgcttccacgagctgggcgacgtggtggacgcc

SEQ ID NO: 11 - DNA, 101 bp, Artificial Sequence
  Description of Artificial Sequence Synthetic primer
tgggcgacgtggtggacgcctttgtggtgtgcgagtccaacttcacggcttatggggagccgcggccgctcaagttccgggagatgctgaccaatggcacc

SEQ ID NO: 12 - DNA, 63 bp, Artificial Sequence
  Description of Artificial Sequence Synthetic primer
agatgctgaccaatggcaccttcgagtacatccgccacaaggtgctctatgtcttcctggacc

SEQ ID NO: 13 - DNA, 70 bp, Artificial Sequence
  Description of Artificial Sequence Synthetic primer
gctctatgtcttcctggaccactttcctcctggaggacgacaagatggatggatcgccgacgactacctg

SEQ ID NO: 14 - DNA, 37 bp, Artificial Sequence
  Description of Artificial Sequence Synthetic primer
tttaagcttactagacttccgcctcgtccagttttcc

SEQ ID NO: 15 - DNA, 109 bp, Artificial Sequence
  Description of Artificial Sequence Synthetic primer
ctagacttccgcctcgtccagttttccccgagcaggcggtcttccttcaggacccctgtggcgccatcctcccgcagccgtgctcctgggctcctggtaggggttgtcc

SEQ ID NO: 16 - DNA, 102 bp, Artificial Sequence
  Description of Artificial Sequence Synthetic primer
ggctcctggtaggggttgtccagaaggtagtggaaccggtcgtagttcttcagcaggtacttgggcgcatacatgtgctcgctggggtctgcaggcgggtac

SEQ ID NO: 17 - DNA, 104 bp, Artificial Sequence
  Description of Artificial Sequence Synthetic primer
ctggggtctgcaggcgggtactcttgctgcgtgccgtcgaaccagcccccggtgcggatcaggccgcggatgtagttcaggtcccgcttgtcctcgtagtcacc

SEQ ID NO: 18 - DNA, 101 bp, Artificial Sequence
  Description of Artificial Sequence Synthetic primer
ccgcttgtcctcgtagtcaccccagcgtgggaagtcgccattctgggcggacacgagcttgaagtagatgccctcgggcgtgaagcaccaggagcagtgcc

SEQ ID NO: 19 - DNA, 102 bp, Artificial Sequence
  Description of Artificial Sequence Synthetic primer
tgaagcaccaggagcagtgccagccggcgaagtgaagggggctgcccagcgaccactgcaccaggatgtgtccggtgcggttctcatactgtctgaagttgg

SEQ ID NO: 20 - DNA, 99 bp, Artificial Sequence
  Description of Artificial Sequence Synthetic primer
ctcatactgtctgaagttgggcatggtgtagtattggcggcggcgcaggcggatgccgtccagcccatacactgcctgcagcatgtccaccgtgcagcc

SEQ ID NO: 21 - DNA, 100 bp, Artificial Sequence
  Description of Artificial Sequence Synthetic primer
agcatgtccaccgtgcagcctgacaccacctccagggtgcccggttgcttccaaaagaatccgtagagcgacgtgcgcatgtggaaggcgaagggctcgg

SEQ ID NO: 22 - DNA, 102 bp, Artificial Sequence
  Description of Artificial Sequence Synthetic primer
gtggaaggcgaagggctcggtccagccatcgtagagcttgaggaacaggacgccgtcacgggccgggatctcgtccgcatcgtcaatgatgaagacgtcgtc

SEQ ID NO: 23 - DNA, 91 bp, Artificial Sequence
  Description of Artificial Sequence Synthetic primer
tcaatgatgaagacgtcgtcgggccgcaggttgcgcagccgcgagacgccgtcctgggtgaggaaggtgcgcaggtagtcgtcggcgatcc

SEQ ID NO: 24 - DNA, 70 bp, Artificial Sequence
  Description of Artificial Sequence Synthetic primer
caggtagtcgtcggcgatccatccatcttgtcgtcctccaggaggaaagtggtccaggaagacatagagc

SEQ ID NO: 25 - DNA, 81 bp, Artificial Sequence
  Description of Artificial Sequence Synthetic primer
ggcggtctctccagcatcttggtgccgggtttgaagcagacgcctccagccttggtgcgcacgaaatactcggtggtgtcc

SEQ ID NO: 26 - DNA, 80 bp, Artificial Sequence
  Description of Artificial Sequence Synthetic primer
acgaaatactcggtggtgtcctcgggcagcaccaagtccacccggtggagctcctcggccgccttgctgggcggcagcgg

SEQ ID NO: 27 - DNA, 68 bp, Artificial Sequence
  Description of Artificial Sequence Synthetic primer
tttggatccgttggccccctcaggcttctcctccggtcgtcccggaggcggtctctccagcatcttgg

SEQ ID NO: 28 - DNA, 1596 bp, Homo sapiens
atgagacgctacaagctctttctcatgttctgtatggccggcctgtgcctcatctccttcctgcacttcttcaagaccctgtcctatgtcaccttcccccgagaactggcctccctcagccctaacctggtgtccagctttttctggaacaatgccccggtcacgccccaggccagccccgagccaggaggccctgacctgctgcgtaccccactctactcccactcgcccctgctgcagccgctgccgcccagcaaggcggccgaggagctccaccgggtggacttggtgctgcccgaggacaccaccgagtatttcgtgcgcaccaaggccggcggcgtctgcttcaaacccggcaccaagatgctggagaggccgcccccgggacggccggaggagaagcctgagggggccaacggctcctcggcccggcggccaccccggtacctcctgagcgcccgggagcgcacggggggccgaggcgcccggcgcaagtgggtggagtgcgtgtgcctgcccggctggcacggacccagctgcggcgtgcccactgtggtgcagtactccaacctgcccaccaaggagcggctggtgcccagggaggtgccgcgccgcgtcatcaacgccatcaacgtcaaccacgagttcgacctgctggacgtgcgcttccacgagctgggcgacgtggtggacgcctttgtggtgtgcgagtccaacttcacggcttatggggagccgcggccgctcaagttccgggagatgctgaccaatggcaccttcgagtacatccgccacaaggtgctctatgtcttcctggaccacttcccgcccggcggccggcaggacggctggatcgccgacgactacctgcgcaccttcctcacccaggacggcgtctcgcggctgcgcaacctgcggcccgacgacgtcttcatcattgacgatgcggacgagatcccggcccgtgacggcgtccttttcctcaagctctacgatggctggaccgagcccttcgccttccacatgcgcacgtcgctctacggcttcttctggaagcagccgggcaccctggaggtggtgtcaggctgcacggtggacatgctgcaggcagtgtatgggctggacggcatccgcctgcgccgccgccagtactacaccatgcccaacttcagacagtatgagaaccgcaccggccacatcctggtgcagtggtcgctgggcagccccctgcacttcgccggctggcactgctcctggtgcttcacgcccgagggcatctacttcaagctcgtgtccgcccagaatggcgacttcccacgctggggtgactacgaggacaagcgggacctgaactacatccgcggcctgatccgcaccgggggctggttcgacggcacgcagcaggagtacccgcctgcagaccccagcgagcacatgtatgcgcccaagtacctgctgaagaactacgaccggttccactacctgctggacaacccctaccaggagcccaggagcacggcggcgggcgggtggcgccacaggggtcccgagggaaggccgcccgcccggggcaaactggacgaggcggaagtctag

SEQ ID NO: 29 - DNA, 1596 bp, Homo sapiens
atgagacgctacaagctctttctcatgttctgtatggccggcctgtgcctcatctccttcctgcacttcttcaagaccctgtcctatgtcaccttcccacgagaactggcctccctcagccctaacctggtgtccagctttttctggaacaatgccccggtcacgccccaggccagccctgagccaggaggccctgacctgctgcgtaccccactctactcccactcgcccctgctgcagccgctgccgcccagcaaggcggccgaggagctccaccgggtggacttggtgctgcccgaggacaccaccgagtatttcgtgcgcaccaaggctggaggcgtctgcttcaaacccggcaccaagatgctggagagaccgcctccgggacgaccggaggagaagcctgagggggccaacggatcctcggcccggcgaccaccccggtacctcctgagcgcccgggagcgcacggggggccgaggtgcacgacgcaagtgggtggagtgcgtgtgtctgcccggatggcacggacccagctgcggcgtgcccactgtggtgcagtattccaacctgcctaccaaggagcggctggtgcccagggaggtgccgcgccgcgtcattaatgctatcaacgtcaaccacgagttcgacctgctggacgtgcgcttccacgagctgggcgacgtggtggacgcctttgtggtgtgcgagtccaacttcacggcttatggggagccgcggccgctcaagttccgggagatgctgaccaatggcaccttcgagtacatccgccacaaggtgctctatgtcttcctggaccactttcctcctggaggacgacaagatggatggatcgccgacgactacctgcgcaccttcctcacccaggacggcgtctcgcggctgcgcaacctgcggcccgacgacgtcttcatcattgacgatgcggacgagatcccggcccgtgacggcgtcctgttcctcaagctctacgatggctggaccgagcccttcgccttccacatgcgcacgtcgctctacggattcttttggaagcaaccgggcaccctggaggtggtgtcaggctgcacggtggacatgctgcaggcagtgtatgggctggacggcatccgcctgcgccgccgccaatactacaccatgcccaacttcagacagtatgagaaccgcaccggacacatcctggtgcagtggtcgctgggcagcccccttcacttcgccggctggcactgctcctggtgcttcacgcccgagggcatctacttcaagctcgtgtccgcccagaatggcgacttcccacgctggggtgactacgaggacaagcgggacctgaactacatccgcggcctgatccgcaccgggggctggttcgacggcacgcagcaagagtacccgcctgcagaccccagcgagcacatgtatgcgcccaagtacctgctgaagaactacgaccggttccactaccttctggacaacccctaccaggagcccaggagcacggctgcgggaggatggcgccacaggggtcctgaaggaagaccgcctgctcggggaaaactggacgaggcggaagtctag

(19) United States
(12) Patent Application Publication
(10) Pub. No.: US 2007/0142627 A1
(43) Pub. Date: Jun. 21, 2007

(54) Sugar-chain-altered anti-hm1.24 antibody

(75) Inventors: Masayuki Tsuchiya, Shizuoka (JP); Shigeyuki Iijima, Shizuoka (JP); Izumi Sugo, Shizuoka (JP); Masamichi Sugimoto, Kanagawa (JP)

Correspondence Address: FOLEY AND LARDNER LLP;SUITE 500, 3000 K STREET NW, WASHINGTON, DC, 20007, US

(73) Assignee: CHUGAI SEIYAKU KABUSHIKI KAISHA

(21) Appl. No.: 10/567,856

(22) PCT Filed: Aug. 11, 2004
(86) PCT No.: PCT/JP04/11812

§ 371 Date: Feb. 10, 2006

(30) Foreign Application Priority Data
Aug. 1, 2003 (JP) 2003-207165

(51) Int. Cl. C12P 21/06; C07K 16/46

(52) U.S. Cl. 530/391.1; 530/387.1; 435/69.1

(57) ABSTRACT

An antibody (anti-HM1.24 antibody) against HM1.24 antigen in which the alteration of sugar chains resulted in enhanced antibody-dependent cellular cytotoxicity (ADCC), specifically an antibody having a sugar chain that does not contain α-1,6 core fucose, an antibody having a sugar chain that contains a bisecting N-acetylglucosamine (GlcNAc) structure, or an antibody having a sugar chain that does not contain α-1,6 core fucose and having a sugar chain that contains a bisecting N-acetylglucosamine (GlcNAc) structure.

### FIELD OF THE INVENTION

The present invention relates to an antibody (anti-HM1.24 antibody) against HM1.24 antigen, said antibody having an enhanced antibody-dependent cellular cytotoxicity (ADCC), and a method of producing said antibody.

### BACKGROUND ART

HM1.24 antigen is a membrane protein with a molecular weight of 29-33 kDa that is strongly expressed on the surface of myeloma cells (Ishikawa J. et al., Genomics 26 (1995), 527-534). The expression has also been noted in B tumor cells and T tumor cells, in addition to myeloma cells. In normal cells, the expression has been confirmed in immunoglobulin-producing B cells and activated T cells but little expression has been noted in other cells (Goto T. et al., Blood 84 (1994), 1922-1930).

As antibody (anti-HM1.24 antibody) against HM1.24 antigen is specifically accumulated in tumors due to the above tissue distribution of HM1.24 antigen, the antibody is very promising in applications such as the diagnosis of tumor localization and missile therapies such as radioimmunotherapy by radiolabelling the antibody and, as the antibody per se has antibody-dependent cellular cytotoxicity (ADCC) (Ozaki S. et al., Blood 90 (1997), 3179-3186), its use as a therapeutic agent for a myeloma such as multiple myeloma is promising.

In such therapeutic applications of anti-HM1.24 antibody, it is preferred to have a low immunogenicity to humans, and to this end, reshaped human (humanized) antibody of anti-HM1.24 antibody has been developed (WO 98/14580). However, it has been desired to provide a HM1.24 antibody that has a further enhanced ADCC activity and, specifically, a humanized HM1.24 antibody that has an enhanced ADCC activity.

As a method of enhancing the ADCC activity of antibody, alteration of sugar chains of antibody has been known. WO 99/54342, for example, describes modifying the glycosylation of antibody to improve ADCC activity. WO 00/61739 also describes the regulation of ADCC activity by the presence or absence of fucose in antibody sugar chains. WO 02/31140 describes the preparation of an antibody having no α-1,6 core fucose by producing the antibody in the YB2/0 cell. WO 02/79255 describes an antibody having a sugar chain that comprises bisected GlcNAc. However, no HM1.24 antibody has been known in which ADCC activity has been enhanced by the modification of sugar chains.

Patent document 1: WO 98/14580

Patent document 2: WO 099/54342

Patent document 3: WO 00/61739

Patent document 4: WO 02/31140

Patent document 5: WO 02/79255

Non-patent document 1: Ishikawa J. et al., Genomics 26 (1995), 527-534

Non-patent document 2: Goto T. et al., Blood 84 (1994), 1922-1930

Non-patent document 3: Ozaki S. et al., Blood 90 (1997), 3179-3186

### DISCLOSURE OF THE INVENTION

Thus, it is an object of the present invention to provide an anti-HM1.24 antibody having an enhanced ADCC activity by the modification of sugar chains, and a method of producing said antibody.

After intensive and extensive study to resolve the above problems, the present inventors have found that an anti-HM1.24 antibody having a sugar chain that contains no α-1,6 core fucose (position 6 of N-acetylglucosamine at reducing end and position 1 of fucose are α-bonded), and an antibody having a sugar chain that has a bisecting N-acetylglucosamine (GlcNAc) structure have a high ADCC activity, and that an anti-HM1.24 antibody having both a sugar chain that contains no α-1,6 core fucose and a sugar chain that has a bisecting N-acetylglucosamine (GlcNAC) structure has a further higher ADCC activity, and therefore have completed the present invention.

Thus, the present invention provides a sugar chain-altered antibody (anti-HM1.24 antibody) against HM1.24 antigen, for example an antibody (anti-HM1.24 antibody) against HM1.24 antigen wherein antibody-dependent cellular cytotoxicity (ADCC) has been enhanced by altering a sugar chain of said antibody. The antibody is typically a monoclonal antibody or an antibody derived therefrom, such as a chimeric antibody, or more preferably a humanized antibody. More specifically, the present invention provides an antibody having a sugar chain that contains no α-1,6 core fucose, an antibody having a sugar chain that has a bisecting N-acetylglucosamine (GlcNAc) structure, and furthermore an antibody having both a sugar chain that contains no α-1,6 core fucose and a sugar chain that has a bisecting N-acetylglucosamine (GlcNAc) structure.

The present invention also provides an antibody composition comprising an anti-HM1.24 antibody having a fucose-free sugar chain and wherein the relative ratio of the fucose-free sugar chain is 30% or more. More preferably, such a relative ratio is 35% or more.

The present invention also provides a method of producing said sugar chain-altered antibody which method comprises culturing YB2/0 cells having introduced therein a nucleic acid encoding an antibody (anti-HM1.24 antibody) against HM1.24 antigen, and harvesting said antibody from said culture; which method comprises culturing a host cell having introduced therein a nucleic acid encoding N-acetylglucosaminyl transferase III (GnTIII), and harvesting said antibody from said culture; and which method comprises culturing YB2/0 cells having introduced therein a nucleic acid encoding N-acetylglucosaminyl transferase III (GnTIII), and harvesting said antibody from said culture.

### BRIEF EXPLANATION OF THE DRAWINGS

FIG. 1 shows a SDS-PAGE (12% T) pattern of purified humanized anti-human HM1.24 antibody expressed in YB2/0. The left figure: under a reducing condition, the right figure: under a non-reducing condition. Four μg of purified humanized anti-human HM1.24 antibody was applied.

FIG. 2 is a result of an experiment in which the ADCC activity of human PEMC was determined at each concentration of HM1.24 antibody-DG44 and HM1.24 antibody-YB with four types of HM1.24 antigen-expressing CHO cells (HM26, HM31, HM21, HM36) as the target cell at an E/T ratio=25.

FIG. 3 is a result of an experiment in which the ADCC activity of human PBMC was determined at 1 mg/ml of HM1.24 antibody-DG44 and HM1.24 antibody-YB with HM31 as the target cell at an E/T ratio=1, 5, and 25.

FIG. 4 is a chromatogram of reverse phase HPLC of a pyridylaminated sugar chain prepared from a CHO-derived antibody (a) and a YB2/0-derived antibody (b). It shows that sugar chain patterns are different with different types of producing cells, and specifically in the YB2/0-derived antibody, a group of peaks (A-D) estimated to be fucose-free are increased.

FIG. 5 shows the structures of sugars A-H shown in FIG. 4 and Table 1.

FIG. 6 shows the structures of sugars I-O shown in FIG. 4 and Table 1.

FIG. 7 shows the combination of human GnTIII CDNA with primer sequences used for total synthesis by PCR. PCR fragments with flanking the BamHI sequence that had previously been introduced into primers were ligated at the same sites to obtain the total sequence of human GnTII cDNA.

FIG. 8 is a comparison of ADCC activity at 100 ng/ml of HM1.24 antibody-DG44 and antibodies derived from clones that produce GnTIII-expressing and producing humanized anti-human HM1.24 antibody. By allowing GnTIII to be expressed, antibody-producing clones having an enhanced ADCC activity were obtained.

FIG. 9 is a result of an experiment in which the ADCC activity of human PBMC was determined at each concentration of HM1.24 antibody-DG44 and humanized anti-human HM1.24 antibody derived from GnTIII-expressing CHO cells with HM36 as the target cell at an E/T ratio=25.

FIG. 10 shows a comparison of a nucleotide sequence (SEQ ID NO: 28) (GnTIII ori.nuc) encoding the native human GnTIII with a nucleotide sequence (SEQ ID NO: 29) (GnTIII mut.nuc) encoding a mutant human GnTIII. In the figure, the asterisk indicates that the corresponding nucleotides in both sequences are the same.

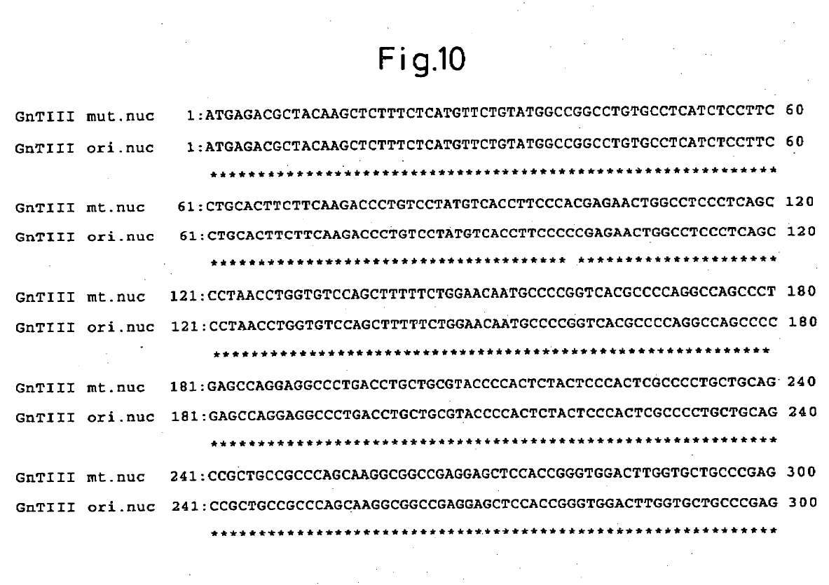

FIG. 11 shows a comparison of a nucleotide sequence (SEQ ID NO: 28) (GnTIII ori.nuc) encoding the native human GnTIII with a nucleotide sequence (SEQ ID NO: 29) (GnTIII mut.nuc) encoding a mutant human GnTIII. In the figure, the asterisk indicates that the corresponding nucleotides in both sequences are the same.

FIG. 12 shows a comparison of a nucleotide sequence (SEQ ID NO: 28) (GnTIII ori.nuc) encoding the native human GnTIII with a nucleotide sequence (SEQ ID NO: 29) (GnTIII mut.nuc) encoding a mutant human GnTIII. In the figure, the asterisk indicates that the corresponding nucleotides in both sequences are the same.

FIG. 13 shows a comparison of a nucleotide sequence (SEQ ID NO: 28) (GnTIII ori.nuc) encoding the native human GnTIII with a nucleotide sequence (SEQ ID NO: 29) (GnTIII mut.nuc) encoding a mutant human GnTIII. In the figure, the asterisk indicates that the corresponding nucleotides in both sequences are the same.

FIG. 14 shows a comparison of a nucleotide sequence (SEQ ID NO: 28) (GnTIII ori.nuc) encoding the native human GnTIII with a nucleotide sequence (SEQ ID NO: 29) (GnTIII mut.nuc) encoding a mutant human GnTIII. In the figure, the asterisk indicates that the corresponding nucleotides in both sequences are the same.

FIG. 15 shows a comparison of a nucleotide sequence (SEQ ID NO: 28) (GnTIII ori.nuc) encoding the native human GnTIII with a nucleotide sequence (SEQ ID NO: 29) (GnTIII mut.nuc) encoding a mutant human GnTIII. In the figure, the asterisk indicates that the corresponding nucleotides in both sequences are the same.

### BEST MODE FOR CARRYING OUT THE INVENTION

In accordance with the present invention, antibody (sugar chain-altered antibody) of which sugar chain has been altered means an antibody that, when compared with sugar chain of the antibody (preferably the antibody that is produced in the highest proportion among the antibodies produced by such a reference host cell) produced by the reference host cell, has a different sugar chain structure, and comprises antibodies having sugar chain structures that are not normally (or predominantly) produced by the reference host cell. Also, when the sugar chain-altered antibodies of the present invention are considered an assembly (also referred to herein as the antibody composition) of antibody molecules having various sugar chains that not always uniform, it means an antibody composition having different proportions of sugar chain-altered antibodies as compared to the antibody composition produced by the reference host cell, and such antibody composition of the present invention also includes antibody compositions that contain antibodies having sugar chain structures that are not normally (or predominantly) produced by the reference host cell.

The reference host cell includes, but is not limited to, CHO dhfr- cells (ATCC CRL-9096), CHO K1 (ATCC CCL-61), CHO DG44 and the like, and preferably the CHO DG44 cell is used as the reference host cell.

As examples of sugar chain-altered antibodies, there can be mentioned fucose-deficient antibodies having sugar chains in which fucose (preferably α-1,6 core fucose) is deficient, antibodies in which bisecting N-acetylglucosamine (GlcNAc) has been added to the sugar chain thereof, and the like.

When the sugar chain-altered antibody of the present invention is considered as an antibody composition, it does not need to be a composition that contains homogeneous antibodies having sugar chains of the identical structure, and thus, in accordance with the present invention, the proportion of the sugar chain-altered antibodies contained in the antibody composition only needs to be different from that of the sugar chain-altered antibodies contained in the antibody composition produced by the above reference host cell. The present invention includes compositions of such sugar chain-altered antibodies. The identity of the composition of sugar chain-altered antibodies of the present invention can be confirmed based on whether the proportion is different from that of the sugar chain-altered antibodies contained in the reference antibody composition described above. Such a proportion can be compared by the relative proportion of each sugar chain recognized by the analytical method disclosed in, for example, Working Example 8 described below.

In order to obtain anti-HM1.24 antibody of the present invention in which the ADCC activity has been enhanced by the modification of sugar chains, it is necessary to express anti-HM1.24 antibody in a host cell having no or low ability of adding α-1,6 core fucose or to express anti-HM1.24 antibody in a host cell having an ability of forming a bisecting N-acetylglucosamine (GlcNAc) structure on a sugar chain. To that end, a gene encoding the desired anti-HM1.24 antibody must be cloned. Anti-HM1.24 antibodies encoded by the cloned gene include, for example, monoclonal antibodies, chimeric antibodies in which the variable region is derived from an animal other than the human and the constant region is derived from a human antibody, humanized antibodies in which the complementarity determining region alone of the variable region is derived from an antibody of an animal other than the human and the other regions of the antibody are derived from a human antibody, and the like.

As can be seen from the description in WO 98/14580, the hybridoma that produces the monoclonal anti-HM1.24 antibody has already been established and has been deposited on Apr. 27, 1995 with the Patent Microorganism Depository, the National Institute of Bioscience and Human Technology (Chuo Dai 6, 1-1, Higashi 1-chome, Tsukuba city, Ibaraki Pref., Japan) as FERM BP-5233. From this hybridoma, DNA encoding a light chain variable region (L chain V region) and DNA encoding a heavy chain variable region (H chain V region) have been cloned, and E. coli having the plasmids containing these DNA's have been deposited on Aug. 29, 1996 with the Patent Microorganism Depository, the National Institute of Bioscience and Human Technology (Chuo Dai 6, 1-1, Higashi 1-chome, Tsukuba city, Ibaraki Pref., Japan) as Escherichia coli DH5α (pUC19-1.24L-gκ) (FERM BP-5646) and Escherichia coli DH5α (pUC19-1.24H-gγ1) (FERM BP-5644), respectively. Furthermore, from the above cloned DNA encoding the L chain V region and DNA encoding the H chain V region, chimeric anti-HM1.24 antibody and humanized anti-HM1.24 antibody were created. For the humanized antibody, as shown in Table 1 to Table 4 on pages 37-40 in WO 98/14580, versions a and b were created for the L chain of the humanized antibody and versions a to s were created for the H chain, and from the measurement of antigen-binding activity of humanized antibodies created by combining them, it was confirmed, humanized antibodies comprising the combination of the L chain version a and the H chain version r or s produce a potent antigen-binding activity.

Thus, in accordance with the present invention, various monoclonal antibodies, chimeric antibodies, humanized antibodies etc. described in WO 98/14580 cited above can be used. In addition to the above antibodies, however, there can be used chimeric antibodies, humanized antibodies etc. derived from other monoclonal antibodies against HM1.24. As methods of preparation in such cases, there can be used the one described in, for example, WO 98/14580.

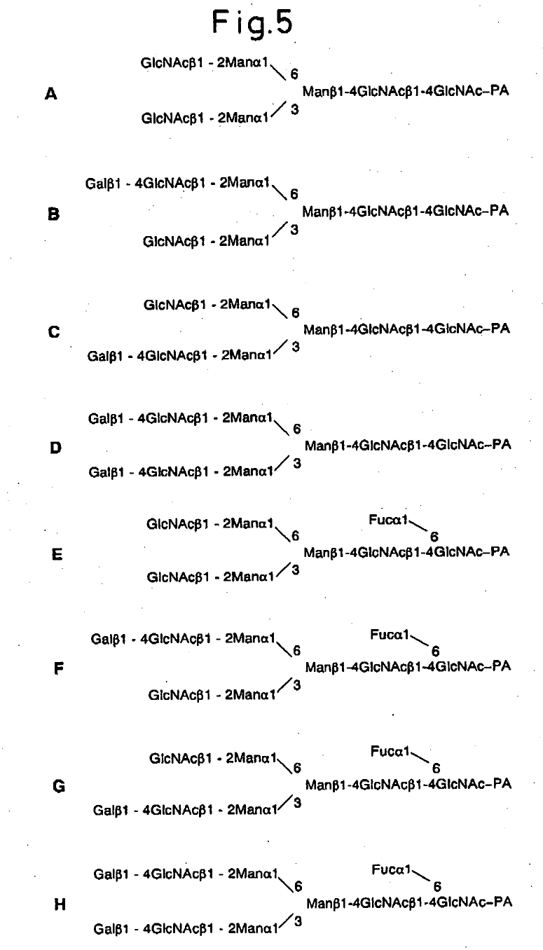

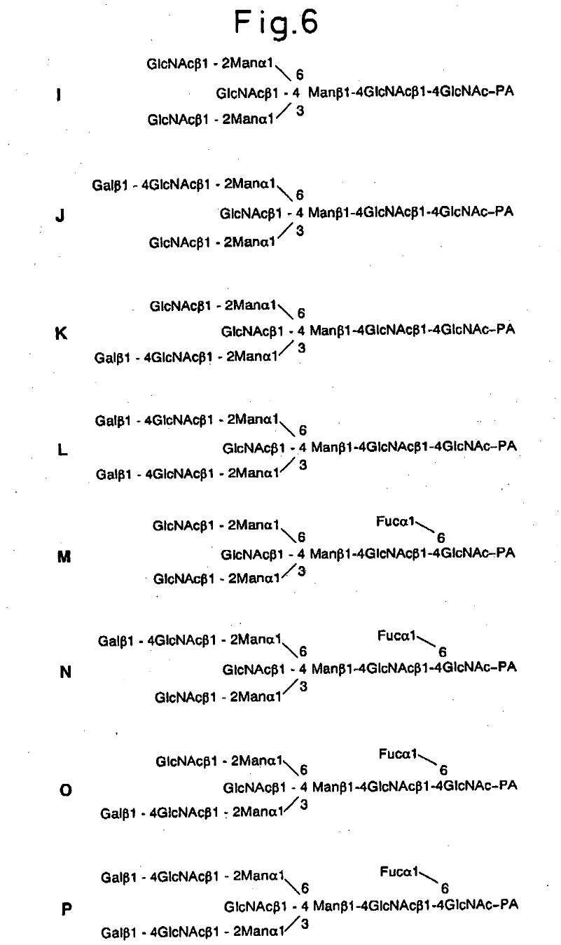

Sugar chains that bind to anti-HM1.24 antibody include N-glycoside-linked sugar chains that bind to the side chain N atom of asparagine of the antibody molecule, and O-glycosyl-linked sugar chains that bind to the side chain hydroxyl group of serine or threonine of the antibody molecule, and the side chains of which presence or absence concerns in the present invention are the N-glycoside-linked sugar chains. The N-glycosyl-linked sugar chains, as shown in FIGS. 5 and 6, have a basic structure (core) [-Manoβ1-4GlcNAcβ1-4GlcNc-] in which one mannose (Man) and two N-acetylglucosamines (GlcNAc) are bound with the β1,4-linkage, and GlNAc on the right of the structure is referred to as the reducing terminal and Man on the left is referred to as the non-reducing terminal. When fucose (Fuc) is bound, N-acetylglucosamine on the position 6 of the reducing terminal and the position 1 of fucose are mainly α-linked.

According to one aspect of the present invention, anti-HM1.24 antibody has a sugar chain that does not contain the above fucose. When an antibody molecule has a plurality of N-glycosyl sugar chains, at least one sugar chain does not have the above fucose. Such an antibody having a fucose-free sugar chain may be produced by expressing said antibody in a cell deficient of an ability of adding fucose to the sugar chain or a host that has no or low fucose-transferring ability.

In accordance with the present invention, any cells that have no or low fucose-transferring ability can be used and, as a specific example, there can be mentioned the rat myeloma YB2/3HL.P2.G11.16Ag.20 cells (abbreviated as the YB2/0 cell) (stored as ATCC CRL 1662). Other cells that can be used in this invention include, for example, the FTVIII knock-out CHO cell (WO 02/31140), and the Lec13 cell (WO 03/035835), the fucose transporter-deficient cell (Patent application No. 2003-174006, Patent application No. 2003-282081, Patent application No. 2003-174010, Patent application No. 2003-282102).

According to another aspect of the present invention, the anti-HM1.24 antibody of the present invention has a sugar chain that has bisecting N-acetylglucosamine. N-glycosyl-linked sugar chains have the basic structure (core) as described above and, on the non-reducing terminal thereof, as shown in FIG. 5, two chains containing mannose are bound with a α1,6-linkage and a α1,3-linkage. On the other hand, in the sugar chain shown in FIG. 6, one N-acetylglucosamine (GlcNAc) is bound with a β1,4-linkage in addition to the above two sugar chains on the non-reducing terminal of the basic structure (core). This N-acetylglucosamine (GlcNAc) is the “bisecting N-acetylglucosamine.”

Sugar chains having bisecting N-acetylglucosamine are O-glycosyl-linked sugar chains or N-glycosyl-linked sugar chains, and are formed by transferring N-acetylglucosamine to sugar chains with N-acetylglucosaminyl transferase III (GnTIII). A gene encoding this enzyme has already been cloned, and the amino acid sequence and the nucleotide sequence of DNA encoding it have been described (NCBI database (ACCESSION D13789)). This DNA can also be cloned according to a standard method such as the PCR method based on the above sequence information.

In order to form sugar chains that have bisecting N-acetylglucosamine using DNA encoding GnTIII, a host cell that produces anti-HM1.24 antibody may be transformed with an expression vector comprising this DNA. Thus, an expression vector comprising DNA encoding GnTIII and an expression vector comprising DNA encoding anti-HM1.24 antibody are used to transform a host cell, which is then cultured.

According to the third aspect of the present invention, the anti-HM1.24 antibody of the present invention has both a sugar chain that does not have α-1,6 core fucose and a sugar chain that has bisecting N-acetylglucosamine. In order to produce this type of antibody, an expression vector comprising DNA encoding GnTIII and an expression vector comprising DNA encoding anti-HM1.24 antibody are used to transform a host cell, such as the YB2/0 cell, that has no or weak activity of forming a sugar chain having α-1,6 core fucose, and then the cell is cultured.

Transformation of host cells, culturing, and isolation and purification of antibody from the culture may be carried out according to standard methods.

Preferably, the sugar chain-altered antibody of the present invention has an enhanced ADCC activity. With regard to whether ADCC activity has been enhanced or not, in accordance with the present invention, ADCC activity is judged to be enhanced when it is higher than that of an antibody in which sugar chains have not been altered or an antibody composition in which sugar chains have not been altered.

ADCC activity may be determined by a method known to a person skilled in the art. In particular, it can be determined by mixing, for example, an effector cell, a target cell and anti-HM1.24 antibody, and then determining the degree of ADCC thereof. More specifically, for example, mouse splenocytes or mononuclear cells derived from human peripheral blood or bone marrow may be used as the effector cell, and CHO cells that express HM1.24 antigen may be used as the target cell. To the target cells that had previously been labelled with ⁵¹Cr, effector cells are added at an appropriate ratio to the target cells, which are then incubated. After incubation, the supernatant is harvested, and radioactivity in the supernatant may be counted to determine ADCC activity.

### EXAMPLES

The present invention will now be explained more specifically with reference to the following examples.

### Example 1

### Expression of Humanized Anti-Human HM1.24 Antibody in Rat Myeloma YB2/0

Ten μg of a vector expressing humanized anti-human HM1.24 antibody (AHi/N5KG1V-lark, Barnett, R.S. et al., Antibody Production in Chinese Hamster Ovary Cells Using Impaired Selectable Marker. In: Wang, H.Y. & Imanaka, T. (eds) ACS Symposium Series Vol 604: Antibody Expression and Engineering, 27, 1995, WO 98/4580) was introduced into 2×10⁶/0.6 ml PBS(−) of YB2/0 (ATCC CRT-1662) by electroporation at a condition of 1.5 kV and 25 μF. Culturing was carried out at 37° C. in a 5% CO2 incubator.

After 400 μg/ml of Geneticin was added to the RPMI 1640 medium (Gibco) containing 10% FCS for selection, a gene was amplified at a sequentially increasing concentration of 50 nM MTX, 100 nM MTX, and 200 nM MTX. Also 0.5 cells/100 μl/well was plated into a 10% FCS/RPMI 1640 containing 200 nM MTX and 400 μg/ml of Geneticin in a 96-well plate (Falcon) in order to clone cells by the limiting dilution method.

The culture supernatant of the YB2/0 cells into which the gene of humanized anti-human HM1.24 antibody had been introduced was determined by ELISA shown in Example 2.

### Example 2

### Determination of Humanized Anti-HM1.24 Antibody (ELISA Method)

To a 96-well ELISA plate (manufactured by Nunc), soluble HM1.24 antigen diluted to about 100 ng/ml with a coat buffer (100 mmol/L sodium hydrogen carbonate, pH 9.6) was added in portions of 100 μL, and then incubated, at 4° C., overnight or longer. After incubation, 1% BSA-PBS was added at 200 μL/well and then allowed to stand at room temperature for about two hours, and the prepared plate was stored at 4° C. After 1% BSA-PBS was decanted off, each well was washed with Tween-PBS.

Appropriately diluted humanized anti-HM1.24 antibody standard solutions or sample solutions and biotin-labelled humanized anti-HM1.24 antibody diluted to 100 ng/ml were mixed at 1:1, and then aliquoted at 100 μL/well. After incubating for 1 hour at room temperature, each well was washed with Tween-PBS. Avidin-labelled HRP was added to each well, incubated at room temperature for 15 minutes or longer, TMB liquid (manufactured by Sigma) was added thereto at 100 μL/well, and 50 μL/well of 2 mol/L sulfuric acid was added to stop the reaction, and then the absorbance at 450 nm was measured. From the calibration curve of concentration-absorbance for humanized anti-HM1.24 antibody standard solutions, the humanized HM1.24 antibody concentration of sample solutions was calculated.

### Example 3

### Purification of Humanized Anti-Human HM1.24 Antibody Expressed in YB2/0

Cells for which the expression of humanized anti-human HM1.24 antibody was confirmed were subjected to an expansion culture with 1700 cm² of a roller bottle (CORNING). Thus, 1×10⁹ humanized anti-human HM1.24 antibody-expressing YB2/0 cells were cultured to confluence in a 400 ml of 10% FCS/RPMI 1640 medium containing 200 nM MTX and 400 μg/ml of gentamycin (2.5 rpm). Then, FCS was passed through rProtein A FF (Amersham Pharmacia) equalized with PBS(−) for collection of the culture supernatant to remove bovine-derived IgG (FCS(−)), and this FCS(−) was cultured in a 10% FCS(−)/RPMI 1640 medium containing 200 nM MTX and 400 μg/ml of geneticin for 3-4 days.

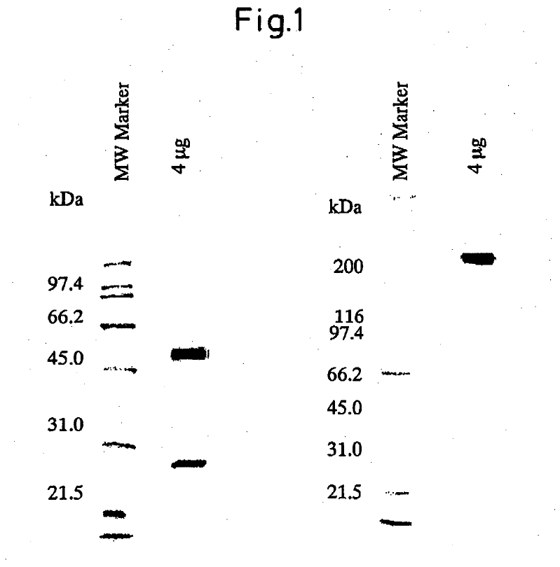

After the culture supernatant was treated with a 0.22 μm filter, it was purified with rProtein A FF (PBS/PBS-citric acid: linear gradient elution) and Source 15S (20 mM acetic acid, 0-0.5 mM NaCl: linear gradient elution). The purified humanized anti-human HM1.24 antibody was termed as HM1.24 antibody-YB (FIG. 1).

### Example 4

### Preparation of CHO Cells Expressing HM1.24 Antigen (BST-2)

CHO cells expressing the HM1.24 antigen protein were prepared as follows (Ohtomo T. et al., Biochemical and Biophysical Research Communications 258 (1999), 583-591). Thus, an expression vector p3.19 (see supra) encoding HM1.24 antigen was introduced to a DHFR-deficient CHO cell line, 500 μg/ml of G418 was used for selection, and the limiting dilution was performed to obtain four cell lines HM26, HM31, HM21 and HM36. The number of the HM1.24 antigen expressed on the cell surface, as determined by flow cytometry in a method described in Patent application No. 2001-115889, was 3.8×10³, 2.2×10⁴, 2.2×10⁴, and 1.8×10⁵, respectively.

### Example 5

### Determination of ADCC Activity Using Human Peripheral Blood-Derived PBMC

(1) Preparation of Human PBMC Solution

Peripheral blood drawn with heparin from a normal healthy subject was diluted by two-fold with PBS (−), and layered on Ficoll-Paque™ PLUS (Amersham Pharmacia Biotech AB). After centrifuging this (500×g, 30 minutes, 20° C.), the interlayer, a mononuclear cell fraction, was collected. After washing three times, it was suspended into 10% PBS/RPMI to prepare the human PBMC solution.

(2) Preparation of the Target Cell Solution

CHO cells that express HM1.24 antigen (BST-2) described in Example 4 were peeled from the dish using the cell detachment buffer (Invitrogen Corp), and were suspended in 200 μl of 10% FBS/RPMI, to which 5.55 MBq of chromium-51 was added, and incubated at 37° C. for one hour in a 5% carbon dioxide incubator. After washing the cells three times, they were prepared into an individual cell concentration in 10% FBS-RPMI 1640 medium to prepare the target cell solutions.

(3) Chromium Release Test (ADCC Activity)

After the target cell solutions were aliquoted in 50 μl portions into a 96-well U-bottomed plate, 50 μl each of antibody solutions prepared at each concentration was added thereto, and incubated on ice for one hour, then 100 μl of the human PBMC solution was added, incubated at 37° C. for four hours in a 5% carbon dioxide incubator, and then the radioactivity of 100 μl of the culture supernatant after incubation was determined by a gamma counter. The specific chromium release rate was determined based on the following formula:
Specific chromium release rate (%)=(A−C)×100/(B−C)

A represents a mean of radioactivity (cpm) for each well, B represents a mean of radioactivity (cpm) of a well in which 50 μl of a target cell suspension, 20 μl of a 10% NP-40 aqueous solution (Nonidet™ P-40, manufactured by Nacalai Tesque Inc.) and 130 μl of a 10% FBS/RPMI medium were added, and C represents a mean of radioactivity (cpm) of a well in which 50 μl of a target cell suspension and 150 μl of a 10% FBS/RPMI medium were added.

### Example 6

### Method of Determining ADCC Activity Using a CHO Cell Line that Stably Expresses β-galactosidase

As the effector cell, mononuclear cells isolated from the peripheral blood of a normal healthy subject by the density centrifugation method was used. Thus, an equal amount of PBS was added to the peripheral blood of the normal healthy subject, layered on Ficoll-Paque PLUS (Pharmacia), and then centrifuged at 500 g for 30 minutes. The mononuclear cell phase was collected, washed three times with RPMI 1640 containing 10% FCS, and then prepared to a cell count of 5×10⁶/ml with α-MEM containing 10% FCS.

After detaching with trypsin-EDTA, 50 μl of the CHO#30 cell line that stably expresses β-galactosidase suspended at 2×10⁵ cells/ml in α-MEM containing 10% FCS and 50 μl of various concentrations of anti-HM1.24 antibody were added into a 96-well U-bottomed plate, which were incubated at 4° C. for 15 minutes. Then 100 μl of the effector cells was added and incubated at 37° C. for four hours. After incubation, 20 μl of the culture supernatant was collected, and the β-galactosidase activity thereof was determined. The maximum amount of the released enzyme was set as the amount of enzyme released with a cell lysis buffer of the Galactone-star assay kit.

Cytotoxic activity was calculated as:
Cytotoxic activity (%)=(A−C)×100/(B−C)
(% β-galactosidase)
wherein, A represents the activity (RLU/sec) of β-galactosidase released in the presence of the antibody, B represents the activity (RLU/sec) of β-galactosidase released with the cell lysis buffer, and C represents the activity (RLU/sec) of β-galactosidase released with the culture liquid alone without antibody.

### Example 7

### Determination of ADCC Activity of YB2/0-Derived Humanized Anti-Human HM1.24 Antibody

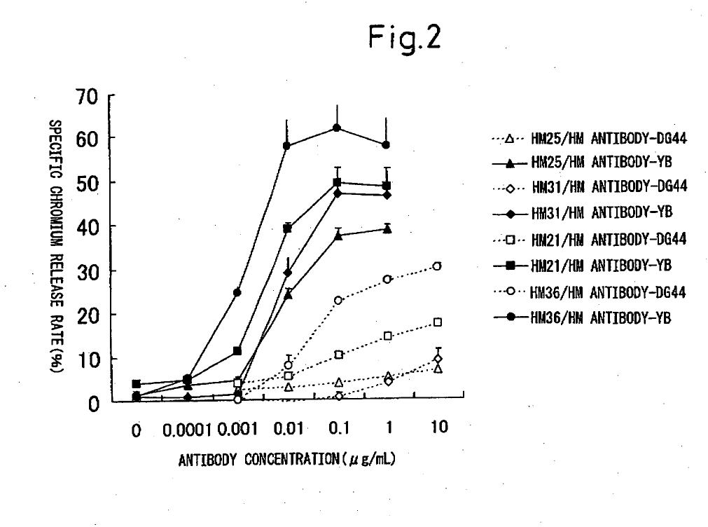

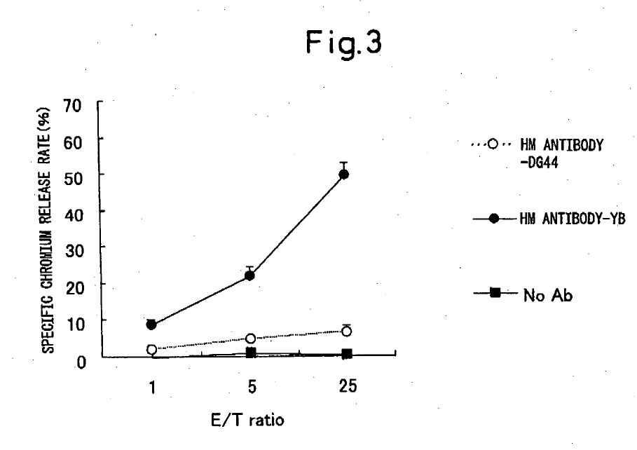

The ADCC activity of HM1.24 antibody (HM1.24 antibody-YB) expressed in YB2/0 determined by the method described in Example 5 is shown in FIG. 2 to FIG. 3. For any target cells, as shown in FIG. 2, HM1.24 antibody-YB exhibited a higher ADCC activity than the HM1.24 antibody (HM1.24 antibody-DG44) produced in DG44 (DHFR-deficient CHO cells: Urlaub, G. et al. (1986) Effect of Gamma Rays at the Dihydrofolate Reductase Locus: Deletions) and Inversions. Somatic Cell and Molecular Genetics, 12: 555, 1986).

Specifically, the induction of ADCC activity was noted at lower concentrations, and the maximal ADCC activity also increased. In particular, when target cells HM26 and HM31 that express small numbers of HM1.24 antigen were used, HM1.24 antibody-DG44 exhibited very low ADCC activity whereas HM1.24 antibody-YB exhibited high ADCC activity. Also, as shown in FIG. 3, not only when the ratio (E/T ratio) of the PBMC count relative to that of the target cells was 25 but when the E/T ratio was 5, HM1.24 antibody-YB exhibited a higher ADCC activity than HM1.24 antibody-DG44.

### Example 8

### Analysis of Sugar Chains

1. Preparation of 2-aminopyridine-Labelled Sugar Chains (Pyridylaminated Sugar Chains)

N-Glycosidase (Roche) reacted to the YB2/0-derived antibody of the present invention and the CHO-derived antibody as the control sample to release sugar chains from the protein (Weitzhandler M. et al., Journal of Pharmaceutical Sciences 83:12 (1994), 1670-1675). After the sugar chains were desalted by solid-phase extraction using a cellulose cartridge (manufactured by TAKARA) (Shimizu Y. et al., Carbohydrate Research 332 (2001), 381-388), they were concentrated to dryness, and then fluorescently labelled with 2-aminopyridine (Kondo A. et al., Agricultural and Biological Chemistry 54: 8 (1990), 2169-2170). After the pyridylaminated sugar chains obtained were desalted by solid-phase extraction using a cellulose cartridge, they were concentrated by centrifugation to prepare purified pyridylaminated sugar chains.

2. Analysis by Reverse Phase HPLC of Purified Pyridylaminated Sugar Chains

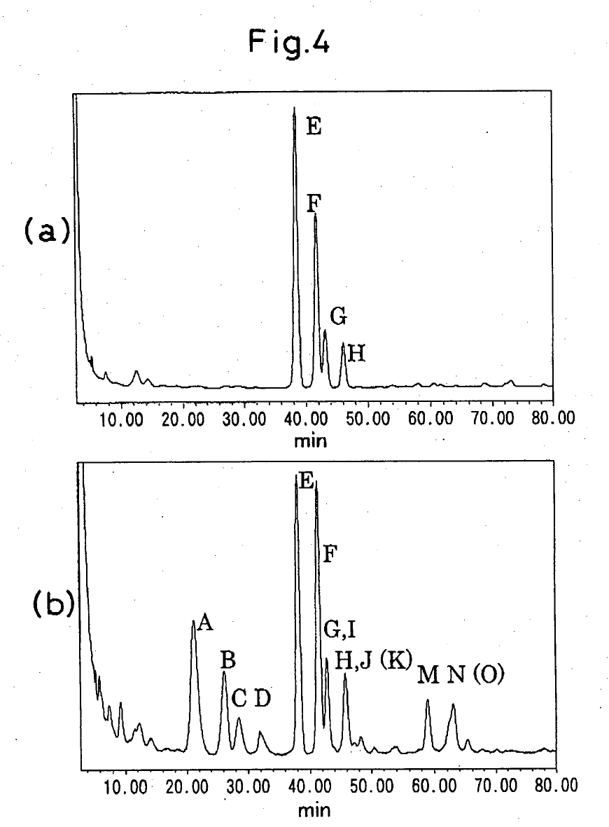

After pyridylaminated sugar chains were prepared for the YB2/0-derived antibody of the present invention and the CHO-derived antibody as the control sample in the method of the above Working Example 8-1, reverse phase HPLC analysis was performed with an ODS column (Palpak Type R manufactured by TAKARA) and the chromatograms were compared. As compared to the sugar chains of the CHO-derived antibody, it was confirmed, the sugar chains of the YB2/0-derived antibody exhibited increases in peaks of sugar chains (A-D), possibly fucose-free, that eluate at 20-35 minutes (FIG. 4).

3. Analysis by Two Dimensional Mapping of Purified Pyridylaminated Sugar Chains

After pyridylaminated sugar chains were prepared for the YB2/0-derived antibody of the present invention in the method of the above Example 8-1, the two dimensional mapping that combined reverse phase HPLC with an ODS column and a normal phase HPLC with an amine column (Palpak Type N manufactured by TAKARA) was performed. Specifically, the normal phase RPLC with an amine column is used to roughly fractionate the purified pyridylaminated sugar chains, and then each fraction is analyzed with the reverse phase HPLC.

Each sugar chain was identified by comparing its elution position in HPLC with those of the pyridylaminated sugar chain standards (manufactured by TAKARA, manufactured by Hohnen, manufactured by Seikagaku Kogyo; excluding K, O, and P in FIG. 5) and confirming molecular weight by TOF-MS. The relative ratio of each sugar chain identified is shown in Table 1 (separation of J from K and separation of N from O have not been performed in this Example). The structures of sugar chains are shown in FIG. 5 and FIG. 6. The result confirmed that in the YB2/0-derived antibody of the present invention, there are 30% or more of fucose-free sugar chains and there are sugar chains that have bisecting GlcNAc.

TABLE 1

| | | Relative ratio | |
|---|---|---|---|
| Sugar | | of each sugar | Relative ratio |
| chain | Group | chain | of each group |
| A | −Fuc, −Bisecting GlcNAc | 17.7% | 33.5% |
| B | | 9.9% | |
| C | | 3.9% | |
| D | | 1.9% | |
| E | +Fuc, −Bisecting GlcNAc | 22.9% | 55.2% |
| F | | 21.4% | |
| G | | 5.5% | |
| H | | 5.4% | |
| I | −Fuc, +Bisecting GlcNAc | 2.0% | 3.3% |
| J(K) | | 1.3% | |
| M | +Fuc, +Bisecting GlcNAc | 3.7% | 8.0% |
| N(O) | | 4.2% | |

### Example 9

### Creation of Human GnTIII-Expressing Expression Vector

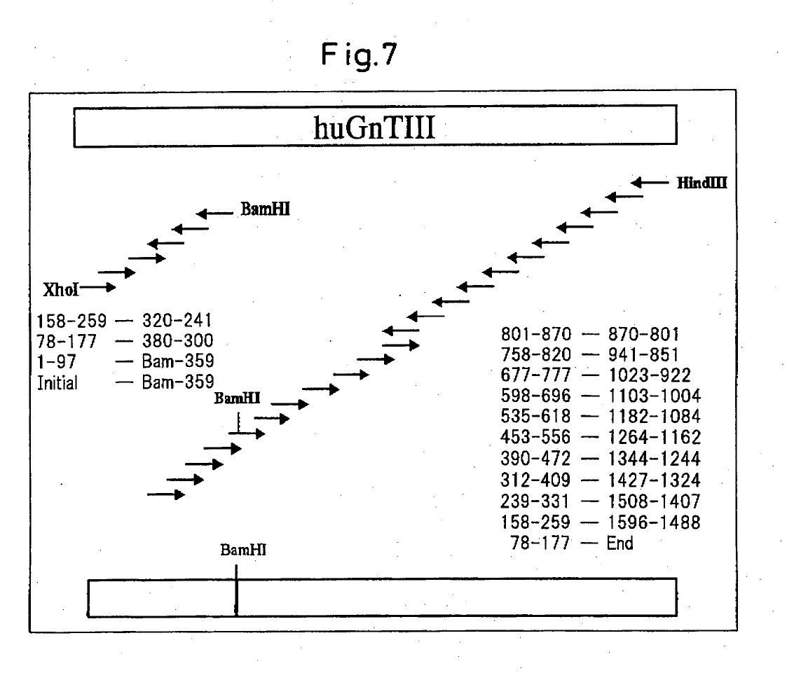

The sequence of the human GnTIII gene was obtained from the NCBI database (ACCESSION D13789). The sequence was analyzed with GENETYX-SV/RC and it was found to have many repeat sequences. In order to facilitate amplification by PCR, primers that had silent mutations in several sites were designed and were obtained by synthesis using PCR. PCR used KOD polymerase (TOYOBO), double strands from the nucleotide numbers 801 to 870 as the initial templates, and the following primers sequentially to perform PCR. In the primer sequences below, bold letters indicate nucleotides in which silent mutations have been introduced. Also, numbers indicate positions from the translation initiation site. FIG. 7 shows the position of each primer relative to the GnTIII gene.

Forward primer

| | |
|---|---|
| Initial (BamHI): | TTTCTCGAGatgagacgctacaagctctttctca |
| | tgttc |
| 1-97: | atgagacgctacaagctctttctcatgttctgta |
| | tggccggcctgtgcctcatctccttcctgcactt |
| | cttcaagaccctgtcctatgtcaccttcc |
| 78-177: | cctgtcctatgtcaccttcccAcgagaactggcc |
| | tccctcagccctaacctggtgtccagctttttct |
| | ggaacaatgccccggtcacgccccaggccagc |
| 158-259: | cggtcacgcccaggccagcccTgagccaggaggc |
| | cctgacctgctgcgtaccccactctactcccact |
| | cgcccctgctgcagccgctgccgccagcaagg |
| 239-331: | agccgctgccgcccagcaaggcggccgaggagct |
| | ccaccgggtggacttggtgctgcccgaggacacc |
| | accgagtatttcgtgcgcaccaagg |
| 312-409: | gtatttcgtgcgcaccaaggcTggAggcgtctgc |
| | ttcaaacccggcaccaagatgctggagagAccgc |
| | cTccgggacgAccggaggagaagcctgagg |
| 390-472: | AccggaggagaagcctgagggggccaacggAtcc |
| | tcggcCcggcgAccaccccggtacctcctgagcg |
| | cccgggagcgcacgg |
| 453-556: | gagcgcccgggagcgcacggggggccgaggTgcA |
| | cgAcgcaagtgggtggagtgcgtgtgTctgcccg |
| | gAtggcacggacccagctgcggcgtgcccactgt |
| | gg |
| 535-618: | agctgcggcgtgcccactgtggtgcagtaTtcca |
| | acctgccTaccaaggagcggctggtgcccaggga |
| | ggtgccgcgccgcgtc |
| 598-696: | agggaggtgccgcgccgcgtcatTaaTgcTatca |
| | acgtcaaccacgagttcgacctgctggacgtgcg |
| | cttccacgagctgggcgacgtggtggacgcc |
| 677-777: | tgggcgacgtggtggacgcctttgtggtgtgcga |
| | gtccaacttcacggcttatggggagccgcggccg |
| | ctcaagttccgggagatgctgaccaatggcacc |
| 758-820: | agatgctgaccaatggcaccttcgagtacatccg |
| | ccacaaggtgctctatgtcttcctggacc |
| 801-870: | gctctatgtcttcctggaccacttTccTccTggA |
| | ggAcgAcaAgaTggAtggatcgccgacgactacc |
| | tg |

Reverse primer

| | |
|---|---|
| End (HindIII): | TTTAAGCTTActagacttccgcctcgtccagtttTc |
| | c |
| 1596-1488: | ctagacttccgcctcgtccagtttTccccgAgcAgg |
| | cggTcttccTtcAggacccctgtggcgccaTccTcc |
| | cgcAgccgtgctcctgggctcctggtaggggttgtc |
| | c |
| 1508-1407: | ggctcctggtaggggttgtccagAaggtagtggaac |
| | cggtcgtagttcttcagcaggtacttgggcgcatac |
| | atgtgctcgctggggtctgcaggcgggtac |
| 1427-1324: | ctggggtctgcaggcgggtactcTtgctgcgtgccg |
| | tcgaaccagcccccggtgcggatcaggccgcggatg |
| | tagttcaggtcccgcttgtcctcgtagtcacc |
| 1344-1244: | ccgcttgtcctcgtagtcaccccagcgtgggaagtc |
| | gccattctgggcggacacgagcttgaagtagatgcc |
| | ctcgggcgtgaagcaccaggagcagtgcc |
| 1264-1162: | tgaagcaccaggagcagtgccagccggcgaagtgAa |
| | gggggctgcccagcgaccactgcaccaggatgtgTc |
| | cggtgcggttctcatactgtctgaagttgg |
| 1182-1084: | ctcatactgtctgaagttgggcatggtgtagtaTtg |
| | gcggcggcgcaggcggatgccgtccagcccatacac |
| | tgcctgcagcatgtccaccgtgcagcc |
| 1103-1004: | agcatgtccaccgtgcagcctgacaccacctccagg |
| | gtgcccggTtgcttccaAaagaaTccgtagagcgac |
| | gtgcgcatgtggaaggcgaagggctcgg |
| 1023-922: | gtggaaggcgaagggctcggtccagccatcgtagag |
| | cttgaggaaCafggacgccgtcacgggccgggatct |
| | cgtccgcatcgtcaatgatgaagacgtcgtc |
| 941-851: | tcaatgatgaagacgtcgtcgggccgcaggttgcgc |
| | agccgcgagacgccgtcctgggtgaggaaggtgcgc |
| | aggtagtcgtcggcgatcc |
| 870-801: | caggtagtcgtcggcgatccaTccAtcTtgTcgTcc |
| | TccAggAggAaagtggtccaggaagacatagagc |
| 380-300 | ggcggTctctccagcatcttggtgccgggtttgaag |
| | cagacgccTccAgccttggtgcgcacgaaatactcg |
| | gtggtgtcc |
| 320-241 | acgaaatactcggtggtgtcctcgggcagcaccaag |
| | tccacccggtggagctcctcggccgccttgctgggc |
| | ggcagcgg |
| Bam-359 | TTTggaTccgttggccccctcaggcttctcctccgg |
| | TcgtcccggAggcggTctctccagcatcttgg |

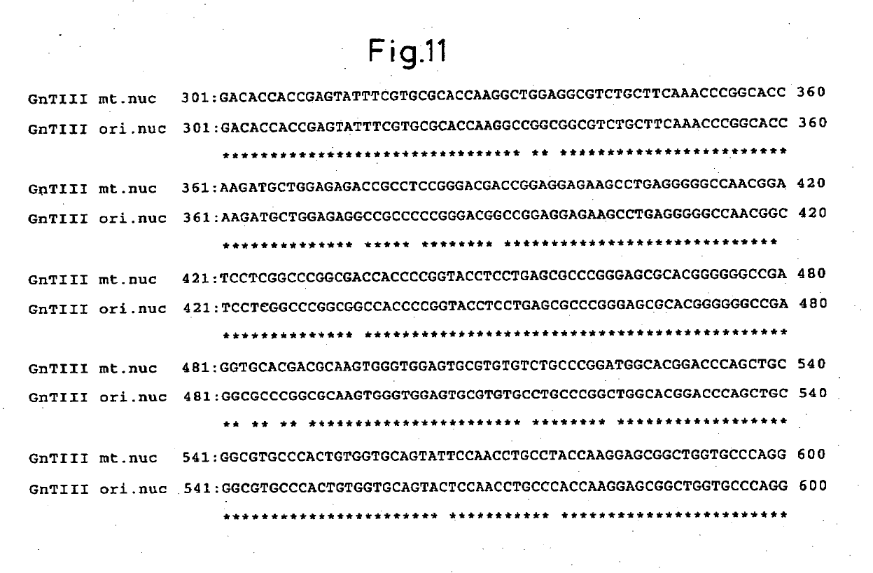

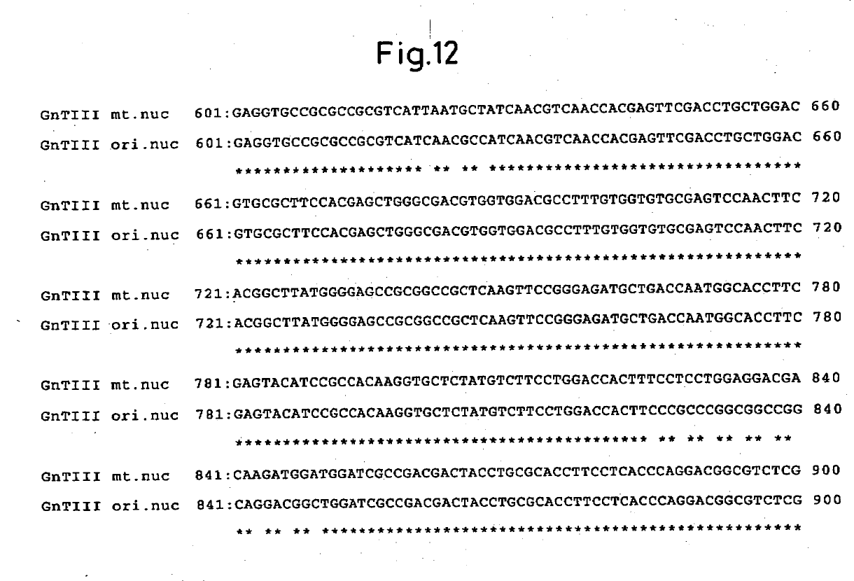

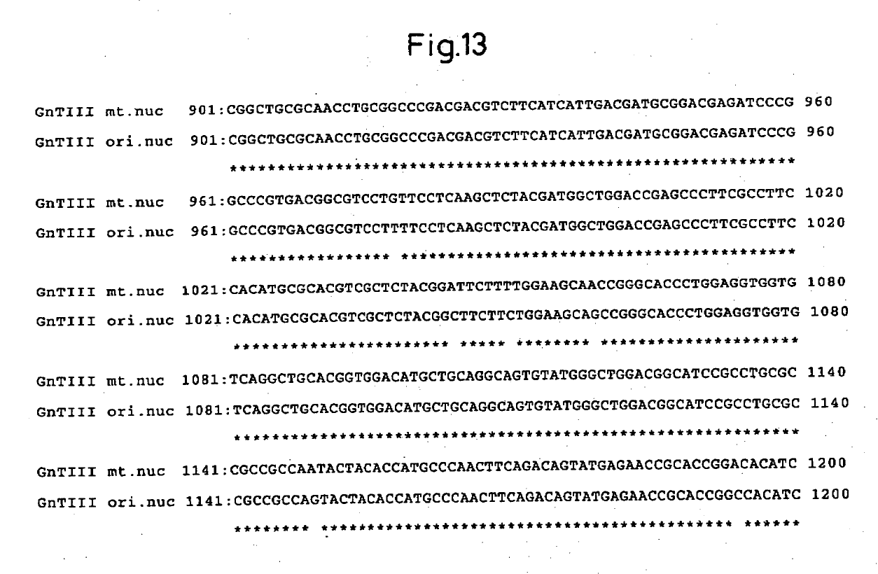

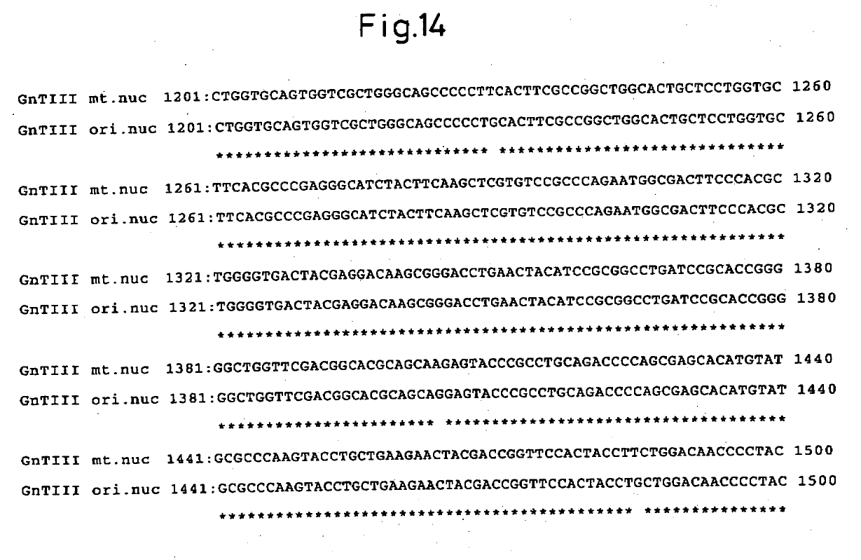

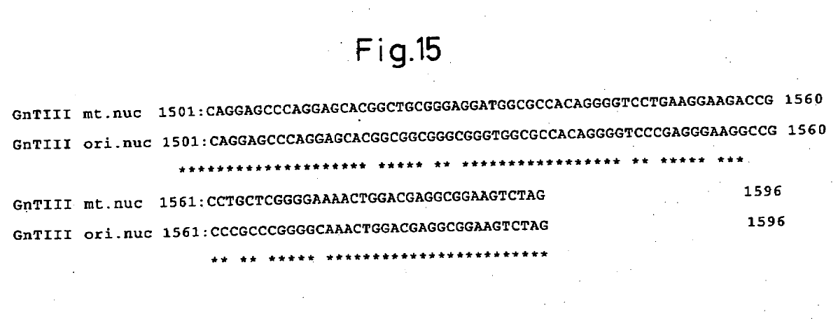

As needed, amplified fragments were subjected to agarose gel electrophoresis, and fragments of interest were excised from the gel and purified, and used as the template for the subsequent PCR. Since it was finally impossible to amplify the full-length by PCR alone, BamHI sites that had previously been introduced into the primer as silent mutation were used, and fragments flanking the sites were ligated after amplification to obtain the full-length human GnTIII gene. FIG. 11 to FIG. 15 show a comparison of a nucleotide sequence (SEQ ID NO: 28) (GnTIII ori.nuc) encoding the native human GnTIII with a nucleotide sequence (SEQ ID NO: 29) (GnTIII mut.nuc) encoding a mutant human GnTIII. In the figure, the asterisk indicates that the corresponding nucleotides in both sequences are the same.

Human GnTIII was integrated into the XhoI/HindIII site of pcDNA3.1 (Hygro-) (Invitrogen) and the sequence was confirmed.

### Example 10

### Expression of GnTIII in CHO Cells that Express HM1.24 Antibody-DG44

Ten μg of GnTIII/pcDNA3.1 (Hygro-) obtained in the above Example 9 was introduced into the HM1.24 antibody-DG44-expressing CHO line by electroporation under a condition of 1.5 kV and 25 μF. Culturing was carried out in a 5% CO2 incubator at 37° C. Using the IMDM medium (Gibco) containing 10% FCS, 10 cells/100 μL/well were plated in a 96-well plate (Falcon), and cultured for two days. The medium was replaced with a 10% FCS/IMDM medium containing 400 μg/ml hygromycin and cells were selected for 1-2 weeks. The culture supernatant of the cells for which hygromycin-resistant colonies developed and growth was noted was collected, and the amount of humanized anti-human HM1.24 antibody was determined by the ELISA method described in Example 2.

### Example 11

### Screening of Humanized Anti-Human HM1.24 Antibody-Producing CHO Cells that Express GnTIII

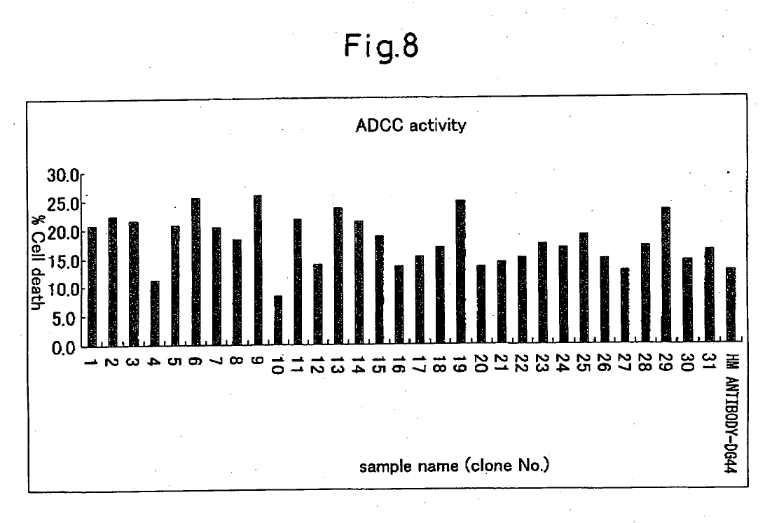

The cultured medium of humanized anti-human HM1.24 antibody derived from humanized anti-human HM1.24 antibody-producing cells (clones No. 1-31) in which GnTIII was forcefully expressed and HM1.24 antibody-DG44 were diluted with the medium to an antibody concentration of 400 ng/ml, and ADCC activity was determined using the method described in Example 6 and compared (FIG. 8).

Finally, screening was carried out taking into account ADCC activity and the amount of humanized anti-human HM1.24 antibody expressed and the growth rate, and the clone No. 6 (57B2) was obtained.

### Example 12

### Determination of ADCC Activity of Humanized Human HM1.24 Antibody Derived From GnTIII-Expressing CHO Cells

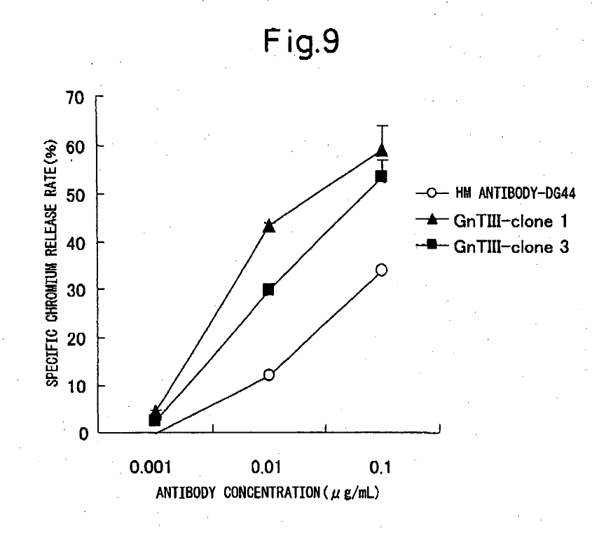

The ADCC activity of humanized anti-human HM1.24 antibody, derived from humanized anti-human HM1.24 antibody-producing cells in which GnTIII was forcefully expressed, was determined in the method described in Example (B) and the result is shown in FIG. 9. Clone No. 3 and No. 6 (57B2) were compared to HM1.24 antibody-DG44 with a result that any of the clones exhibited higher ADCC activity than HM1.24 antibody-DG44.

### INDUSTRIAL APPLICABILITY

In accordance with the present invention, anti-HM1.24 antibody having a high ADCC activity can be produced.

1. A sugar chain-altered antibody (anti-HM1.24 antibody) against HM1.24 antigen.

2. The antibody (anti-HM1.24 antibody) against HM1.24 antigen according to claim 1 in which the alteration of sugar chains resulted in enhanced antibody-dependent cellular cytotoxicity (ADCC).

3. The antibody according to claim 1 in which said antibody is a monoclonal antibody.

4. The antibody according to claim 1 in which said antibody is a chimeric antibody.

5. The antibody according to claim 1 in which said antibody is a humanized antibody.

6. The antibody according to claim 1 having a sugar chain that does not contain α-1,6 core fucose.

7. The antibody according to claim 1 having a sugar chain that contains a bisecting N-acetylglucosamine (GlcNAc) structure.

8. The antibody according to claim 1 having a sugar chain that does not contain α-1,6 core fucose and a sugar chain that contains a bisecting N-acetylglucosamine (GlcNAc) structure.

9. An antibody composition comprising anti-HM1.24 antibody having a fucose-free sugar chain, wherein the relative ratio of the fucose-free sugar chain is 30% or more.

10. A method of producing said antibody according to claim 6 which method comprises culturing cells deficient in fucose-adding ability having introduced therein a nucleic acid encoding an antibody (anti-HM1.24 antibody) against HM1.24 antigen, and harvesting said antibody from said culture.

11. A method of producing said antibody according to claim 7 which method comprises culturing a host cell having introduced therein a nucleic acid encoding N-acetylglucosaminyl transferase III (GnTIII), and harvesting said antibody from said culture.

12. A method of producing said antibody according to claim 8 which method comprises culturing cells deficient in fucose-adding ability having introduced therein a nucleic acid encoding N-acetylglucosaminyl transferase III (GnTIII), and harvesting said antibody from said culture.
